(19) United States
(12) Patent Application Publication
(10) Pub. No.: US 2020/0046716 A1
(43) Pub. Date: Feb. 13, 2020

(54) LAMOTRIGINE SUSPENSION DOSAGE FORM

(71) Applicant: Jubilant Generics Limited, Noida, Uttar Pradesh (IN)

(72) Inventors: Kamal Surendra Mehta, Noida (IN); Dinesh Kumar, Noida (IN); Saurabh Srivastava, Noida (IN); Amit Jha, Noida (IN); Rakesh K. Singh, Noida (IN)

(21) Appl. No.: 16/526,686

(22) PCT Filed: Feb. 2, 2018
(86) PCT No.: PCT/IB2018/050664

§ 371 Date: Jul. 30, 2019

(30) Foreign Application Priority Data
Feb. 3, 2017 (IN) 201711004012

(51) Int. Cl. A61K 31/53; A61K 9/00; A61K 9/14; A61K 47/38; A61K 47/36

(52) U.S. Cl. CPC A61K 31/53; A61K 9/0053; A61K 47/36; A61K 47/38; A61K 9/146

(57) ABSTRACT

The present invention relates to a stable ready to use and powder for oral suspension dosage forms of Lamotrigine and its pharmaceutically acceptable salts and process of preparing such compositions. The liquid and powder for oral suspension dosage forms of Lamotrigine have been previously known only as extemporaneous preparations. Present invention relates to manufacture of liquid and powder for oral suspension dosage forms of Lamotrigine having improved physico-chemical properties with desired technical attributes. The prepared dosage forms are useful in patients having difficulties in swallowing tablets and provide physician with more options for dose titration.

### FIELD OF THE INVENTION

The present invention relates to a pharmaceutical composition in the form of a suspension and suspension powder for reconstitution comprising Lamotrigine. The invention also relates to process for the preparation of such compositions.

### BACKGROUND OF THE INVENTION

Anticonvulsant drugs, particularly drugs from phenyltriazine class are widely used clinically as medicaments for the treatment of epilepsy, bipolar disorder, partial seizures, primary and secondary tonic-clonic seizures, and seizures associated with Lennox-Gastaut syndrome.

Lamotrigine is a prominent anti-epileptic drug of the phenyltriazine group. It is marketed in the form of tablets, chewable/dispersible tablets, orally disintegrating tablets and extended release tablets under the trade name Lamictal® in the USA. The marketed solid dosage forms of Lamotrigine are indicated for the treatment of epilepsy and bipolar disorder.

However, many patients, particularly the elderly and paediatric patients, experience difficulty in swallowing or chewing tablet dosage forms. This has resulted in the widespread practice of crushing tablets to form powder and powder being combined with Ora-sweet and/or Ora-Plus to produce suspension prior to administration. Such extemporaneous preparations have raised concerns over this practice, as they are prone to inaccurate dosing and contamination and harm to the patient.

There is a long-felt need for medicines to be available in liquid form or powder for suspension composition which are suitable for oral administration while maintaining a suitable bioavailability of the drug and/or its active metabolite(s) after oral administration. As such, the present invention aims to address these problems by providing a pharmaceutical composition comprising Lamotrigine or pharmaceutically acceptable salts thereof which is suitable for oral administration in liquid or powder for oral suspension form.

Lamotrigine is BCS class II molecule with low solubility and high permeability. Oral administration is associated with a delayed onset of the desired pharmacological action as lamotrigine is a poorly soluble in water which causes low rate of dissolution of the drug in aqueous media including biological fluids like gastrointestinal fluid. It is also difficult to formulate Lamotrigine into a suspension dosage form due to various challenges like bitter taste of the drug and maintaining the chemical stability of the drug in the suspension dosage form. Further, the formulated suspension should exhibit desirable technical attributes like pourability, viscosity, dissolution, stability, re-suspendability and re-dispersibility complying with demanding requirements and regulations of health and medicine regulatory agencies across the world, especially USFDA, EMEA, Health Canada, MHRA and TGA.

After exhaustive scientific experiments and testing present inventors have surprisingly found that it is possible to develop an oral suspension form of Lamotrigine having improved physical and chemical properties with desired technical attributes for use by patients having difficulties in swallowing tablets.

### OBJECTS AND SUMMARY OF THE INVENTION

It is a principal object of the present invention to provide a stable pharmaceutical composition comprising an anticonvulsant drug with one or more pharmaceutically acceptable excipient and/or carrier and process for its preparation.

It is another object of the present invention to provide a stable pharmaceutical composition in the form of a suspension comprising Lamotrigine or its pharmaceutically acceptable esters, salts, solvates, polymorphs, enantiomers or mixtures thereof with one or more pharmaceutically acceptable excipient and/or carrier and process for their preparation.

It is another object of the present invention to provide an oral pharmaceutical composition in the form of a suspension comprising Lamotrigine or its pharmaceutically acceptable esters, salts, solvates, polymorphs, enantiomers or mixtures thereof with one or more pharmaceutically acceptable excipient and/or carrier including a thickening agent/viscosity agent, antioxidants, anticaking agent, antifoaming agent, pH adjusting agent, coloring agent, sweetening agent, flavouring agent, solubilizer/wetting agent, buffer, diluent, preservative and stabilizer.

Another object of the present invention is to develop a suspension comprising Lamotrigine or its pharmaceutically acceptable esters, salts, solvates, polymorphs, enantiomers or mixtures thereof by a manufacturing process which is consistent and therefore feasible for industrial production, while maintaining stability and pharmaceutical equivalence to the reference formulation(s).

The following embodiments further describe the objects of the present invention in accordance with the best mode of practice, however, disclosed invention is not restricted to the particular embodiments hereinafter described.

In accordance with a one embodiment of the present invention, there is provided a ready to use stable liquid suspension comprising Lamotrigine or its pharmaceutically acceptable esters, salts, solvates, polymorphs, enantiomers or mixtures thereof and at least one or more pharmaceutically acceptable excipient and/or carrier including a thickening agent/viscosity agent, antioxidant, anticaking agent, antifoaming agent, pH adjusting agent, coloring agent, sweetening agent, flavouring agent, solubilizer/wetting agent, buffer, diluent, preservative and stabilizer.

In accordance with another embodiment of the present invention, there is provided dry powder for suspension compositions suitable for use as a liquid suspension for children or elderly patients. The compositions include Lamotrigine or its pharmaceutically acceptable esters, salts, solvates, polymorphs, enantiomers or mixtures thereof and pharmaceutically acceptable excipients selected from the group consisting of suspending agents, viscosity enhancers, coating agents, preservatives, flavouring agents, thickening agent/viscosity agent, sweeteners, lubricants, wetting agents, surfactants, buffering agents, and diluents.

In accordance with still another embodiment of the present invention, there is provided a process for the preparation of a stable pharmaceutical composition in the form of a suspension comprising Lamotrigine or its pharmaceutically acceptable esters, salts, solvates, polymorphs, enantiomers or mixtures thereof, wherein Lamotrigine or its pharmaceutically acceptable esters, salts, solvates, polymorphs, enantiomers or mixtures thereof, wherein liquid carrier including aqueous and/or non-aqueous carrier.

In accordance with still another embodiment of the present invention, there is provided a process for the preparation of a stable pharmaceutical composition in the form of a suspension comprising Lamotrigine or its pharmaceutically acceptable esters, salts, solvates, polymorphs, enantiomers or mixtures thereof, wherein Lamotrigine or its pharmaceutically acceptable esters, salts, solvates, polymorphs, enantiomers or mixtures thereof is in micronized form.

In accordance with another embodiment of the present invention, there is provided a process for the preparation of a stable pharmaceutical composition in the form of a suspension comprising Lamotrigine or its pharmaceutically acceptable esters, salts, solvates, polymorphs, enantiomers or mixtures thereof, is coated to mask bitter taste of the drug.

In accordance with still another embodiment of the present invention, there is provided a process for the preparation of a ready to use liquid suspension of Lamotrigine or its pharmaceutically acceptable esters, salts, solvates, polymorphs, enantiomers or mixtures thereof, comprising combining various components using conventional equipments such as overhead stirrers, ultrasonifiers, mills, homogenizers operating at an RPM range of about 100-8000 RPM, or as per requirement, manufacturing and heating tank with or without vacuum application assembly known in the art or industry practice. Many different orders of adding components to the stirrer can be employed. This can be followed by addition of liquid carrier (such as aqueous and/or non-aqueous), viscosity agent, sweetening agent, taste masking agent and the like. pH of the suspension is adjusted to desired value using aqueous buffering agents as needed.

In accordance with still another embodiment of the present invention, there is provided a process for the preparation of a dry powder for suspension composition of Lamotrigine or its pharmaceutically acceptable esters, salts, solvates, polymorphs, enantiomers or mixtures thereof, which is suitable for suspension in water and/or water miscible suitable solvents to form an orally administerable product which comprises admixing, at ambient temperature and humidity conditions, Lamotrigine granules with substantially dry pharmaceutically acceptable excipients selected from the group consisting of suspending agents, viscosity enhancers, coating agents, preservatives, flavouring agents, sweeteners, viscosity agents, lubricants, wetting agents, surfactants, buffering agents, and diluents to form a dry admixture, and transferring the dry admixture to a sealable storage container.

In accordance with still another embodiment of the present invention, there is provided a stable pharmaceutical composition in the form of a suspension comprising Lamotrigine or its pharmaceutically acceptable esters, salts, solvates, polymorphs, enantiomers or mixtures thereof, wherein the composition is substantially free from other polymorphic forms.

In accordance with still another embodiment of the present invention, there is provided a stable pharmaceutical composition in the form of a suspension comprising Lamotrigine or its pharmaceutically acceptable esters, salts, solvates, polymorphs, enantiomers or mixtures thereof, wherein Lamotrigine has a particle size distribution D₉₀ less than about 200 μm.

In accordance with still another embodiment of the present invention, there is provided a stable pharmaceutical composition in the form of a suspension comprising Lamotrigine or its pharmaceutically acceptable esters, salts, solvates, polymorphs, enantiomers or mixtures thereof, in an amount of about 0.01% to about 90% by weight wherein, the composition exhibits desirable technical attributes like pourability, viscosity, dissolution, stability, re-suspendability and re-dispersibility and a process for preparing the same.

In accordance with still another embodiment of the present invention, there is provided a stable pharmaceutical composition in the form of a suspension comprising Lamotrigine or its pharmaceutically acceptable esters, salts, solvates, polymorphs, enantiomers or mixtures thereof, for the treatment of epilepsy, bipolar disorder, partial seizures, primary and secondary tonic-clonic seizures, and seizures associated with Lennox-Gastaut syndrome.

### DETAILED DESCRIPTION OF THE INVENTION

The present invention can be more readily understood by reading the following detailed description of the invention and study of the included examples.

As used herein, the term “composition or “formulation” or “dosage form”, as in pharmaceutical composition, is intended to encompass a drug product comprising an anticonvulsant or anti-epileptic drug, preferably Lamotrigine or its pharmaceutically acceptable salts, esters, solvates, polymorphs, enantiomers or mixtures thereof, and other inert ingredient(s) (pharmaceutically acceptable excipients). Such pharmaceutical compositions are synonymous with “formulation” and “dosage form”. Pharmaceutical composition of the invention include, but is not limited to, solution, powder for suspension, oral suspension and the like. Preferably, the pharmaceutical composition refers to suspension. More preferably, the pharmaceutical composition refers to ready to use or powder for suspension and suspension powder for reconstitution comprises granules, pellets, or beads.

As used herein, the term “ready to use suspension” means a preconstituted suspension which can be administered as such. The “powder for suspension” or “dry suspension” needs to be reconstituted with a liquid carrier to form a suspension.

As used herein, the term “anticonvulsant or anti-epileptic drug or anti-epileptic drug of phenyltriazine group” is used in broad sense to include not only “anticonvulsant or anti-epileptic drug or anti-epileptic drug of phenyltriazine group ” per se but also its pharmaceutically acceptable salts, pharmaceutically acceptable esters, pharmaceutically acceptable solvates, pharmaceutically acceptable hydrates, pharmaceutically acceptable enantiomers, pharmaceutically acceptable derivatives, pharmaceutically acceptable polymorphs, pharmaceutically acceptable prodrugs, analogs and also its various crystalline and amorphous forms that induce a desired pharmacological or physiological effect. Terms like “active”, “active agent”, “active substance” may be used synonymously for “active ingredient”.

As used herein, the term “Lamotrigine” includes lamotrigine and pharmaceutically acceptable salts, esters, hydrates, solvates, polymorphs, enantiomers, prodrugs, chelates, derivatives, analogs, complexes or mixtures thereof. The pharmaceutical modified release liquid composition of the present invention comprises lamotrigine in an amount from about 0.01% w/w to about 50% w/w of the total composition, particularly in an amount from about 0.01% to about 10% w/w of the total composition.

The term “excipient” means a pharmacologically inactive component such as a thickening agent, viscosity agent, anticaking agent, antifoaming agent, pH adjusting agent, antioxidant, sweetening agent, flavoring agent, solubilizer/wetting agent, buffer, and preservative and the like. The excipients used in preparing the liquid pharmaceutical composition are safe and non-toxic. Reference to an excipient includes both one and more than one such excipient. Co-processed excipients are also covered under the scope of present invention. Further, excipient may be in the form of powders or in the form of dispersion. Combination of excipients performing the same function may also be used to achieve desired formulation characteristics. In addition to the aforementioned components, the Lamotrigine oral suspension form can also optionally contain other excipients commonly found in pharmaceutical compositions such as alternative solvents, taste-masking agents, antioxidants, fillers, acidifiers, enzyme inhibitors and other components as described in Handbook of Pharmaceutical Excipients, Rowe et al., Eds., 6th Edition, Pharmaceutical Press (2009).

“Substantially free” as used herein refers to the pharmaceutical composition of Lamotrigine, which is free from conversion to other polymorphic forms during formulation development or stability studies.

As used herein, the term “about” means±approximately 20% of the indicated value, such that “about 10 percent” indicates approximately 08 to 12 percent.

As used in this specification, the singular forms “a”, “an”, and “the” include plural references unless the context clearly dictates otherwise. Thus, for example, a reference to “a process” includes one or more process, and/or steps of the type described herein and/or which will become apparent to those persons skilled in the art upon reading this disclosure and so forth.

The term “stable,” as used herein, refers to chemical stability, wherein not more than 5% w/w of total related substances are formed on storage at 40° C. and 75% relative humidity (R.H.) or at 25° C. and 60% R.H. for a period of at least one month, particularly for a period of two months, and more particularly for a period of at least three months.

Unless otherwise stated the weight percentages expressed herein are based on the final weight of the composition or formulation.

The present invention is a stable pharmaceutical composition directed to ready to use oral liquid suspension or dry powder for suspension compositions suitable for use as a liquid suspension for administration to a subject in need thereof which comprises Lamotrigine or its pharmaceutically acceptable salts. The suspension dosage form is capable of masking the taste of the drug and also provides the drug in a suitable form to dissolve thereby providing patient compliance, especially for children and the elderly.

In accordance with one embodiment of the present invention, there is provided a stable pharmaceutical composition in the form of a suspension comprising Lamotrigine or its pharmaceutically acceptable salts with one or more pharmaceutically acceptable excipient and/or liquid carrier and process for its preparation.

Another embodiment of the present invention relates to an immediate release oral pharmaceutical suspension dosage form of Lamotrigine or its pharmaceutically acceptable salt, hydrate or polymorph thereof with one or more pharmaceutically acceptable excipients comprising:
a) a viscosity agent/ thickening agent selected from gums and/or celluloses;
b) a diluent and/or sweetening agent selected from sugars and sugar alcohols;
c) a preservative;
d) an antioxidant;
e) pH adjusting agent in sufficient amounts to maintain the pH of the composition in the range of about 4.0 to about 9.0; and/or a pharmaceutically acceptable liquid carrier

wherein composition is free of glidant.

In accordance another embodiment of present invention, there is provided a stable suspension comprising Lamotrigine or its pharmaceutically acceptable salts and one or more pharmaceutically acceptable excipients and/or a liquid carrier, wherein the viscosity agent is selected from the group comprising gums such as xanthan gum, acacia gum, locust bean gum, celluloses, a mixture of carboxymethyl cellulose and microcrystalline cellulose, polyvinyl alcohol and polyvinyl pyrrolidone, colloidal silicon dioxide, carbomer and combinations thereof.

In accordance with one aspect of the present embodiment, there is provided a stable suspension comprising Lamotrigine or its pharmaceutically acceptable salts, wherein the viscosity agent is combination of xanthan gum and a mixture of carboxymethyl cellulose and microcrystalline cellulose.

In accordance with another aspect of the present embodiment, there is provided a stable suspension comprising Lamotrigine or its pharmaceutically acceptable salts, wherein xanthan gum and a mixture of carboxymethylcellulose and microcrystalline cellulose are present in a ratio of 3:1 to 1:3 w/w.

In accordance with another aspect of the present embodiment, there is provided a stable suspension comprising Lamotrigine or its pharmaceutically acceptable salts, wherein the viscosity agent is carrageenan, a mixture of carboxymethylcellulose and microcrystalline cellulose, and combinations thereof.

In accordance with still another aspect of the present embodiment, there is provided a stable suspension comprising Lamotrigine or its pharmaceutically acceptable salts, wherein, the viscosity agent is combination of carrageenan, xanthan gum and a mixture of carboxymethylcellulose and microcrystalline cellulose.

In accordance with another embodiment of the present invention, there is provided a stable pharmaceutical composition in the form of a suspension comprising Lamotrigine or its pharmaceutically acceptable salts and/or a liquid carrier, wherein the diluent is selected from sucrose, sugar alcohol, sorbitol, xylitol, erythritol, starch, pregelatinized starch, calcium carbonate, calcium phosphate, dibasic anhydrous, calcium phosphate, dibasic dihydrate, calcium phosphate tribasic, calcium sulphate, cellulose powdered, silicified microcrystalline cellulose and combinations thereof.

In one aspect of the present embodiment, sucrose has a particle size such that not less than 90% particles are below 200 μm. In particular, sucrose has a particle size such that not less than 90% particles are below 100 μm. This helps in achieving improved uniformity of the drug in the mixture.

In accordance with one embodiment of the present invention, there is provided a stable pharmaceutical composition in the form of a suspension comprising Lamotrigine or its pharmaceutically acceptable salts with one or more pharmaceutically acceptable excipient and/or liquid carrier, wherein liquid carrier is selected from the group comprising aqueous and non-aqueous carrier optionally with one or more pharmaceutically acceptable excipients. The aqueous carrier is selected from the group comprising water or a combination of water and a water-miscible organic solvent. The non-aqueous carrier is selected from the group comprising oils e.g., peanut oil, soy bean oil, corn oil, sesame oil, cottonseed oil; mineral oil; fatty acid esters; mono- or di- fatty acid esters of polyethylene glycols; glyceryl mono-oleate; ethyl oleate; acetylated glycerides; or combinations thereof.

In accordance with another embodiment of the present invention, there is provided a stable pharmaceutical composition in the form of a suspension comprising Lamotrigine or its pharmaceutically acceptable salts and/or a liquid carrier, wherein the sweetening agent is selected from the group sugar or a sugar alcohol such as sucrose, dextrose, sucralose, sorbitol, fructose, mannitol and invert sugar and sugar substitutes such as saccharin sodium, aspartame and combinations thereof.

In accordance with another embodiment of the present invention, there is provided a stable pharmaceutical composition in the form of a suspension comprising Lamotrigine or its pharmaceutically acceptable salts and/or a liquid carrier, wherein the preservative is selected from the group comprising benzoic acid and its salts, sorbic acid and its salts, parabens, sodium metabisulphite, chlorhexidine, sodium citrate, butylated hydroxyl toluene (BHT), butylated hydroxyl anisole (BHA), tocopherol, ethylenediamine tetraacetic acid, propyl gallate and quaternary compounds.

In accordance with another embodiment of the present invention, there is provided a stable pharmaceutical composition in the form of a suspension comprising Lamotrigine or its pharmaceutically acceptable salts and/or a liquid carrier, wherein the antioxidant is selected from the group comprising ascorbic acid, tert-butylhydroquinone, sodium pyrosulfite, glutathione, sodium bisulfite, sodium sulfite, α-tocopherol, α-tocopherol acetate, monothioglycerol, cysteine, ascorbyl palmitate, acetylcysteine, dithiothreitol, sodium metabisulfite, thiourea, and sodium thiosulfate.

In accordance with other embodiment of the present invention, there is provided a stable suspension comprising about 0.01% to about 90% by weight of Lamotrigine or its pharmaceutically acceptable esters, salts, solvates, polymorphs, enantiomers or mixtures thereof, preferably in the range of about 0.1% to about 40% by weight on the basis of the total weight of the composition.

In accordance with other embodiment of the present invention, there is provided a stable suspension comprising Lamotrigine or its pharmaceutically acceptable salts, wherein the amount of Lamotrigine in the suspension ranges from about 0.1 mg/mL to about 400 mg/mL. The amount of Lamotrigine in the suspension ranges preferably from about 0.5 mg/mL to 300 mg/mL, preferably from about 0.5 mg/mL to 200 mg/mL, preferably from about 0.5 mg/mL to 100 mg/mL. More preferably the amount of Lamotrigine in the suspension ranges from about 0.5 mg/mL to 75 mg/mL. In accordance with one aspect of the present embodiment, there is provided a stable suspension comprising Lamotrigine or its pharmaceutically acceptable salts, wherein the amount of Lamotrigine in suspension is 1 mg/mL, 2mg/5mL, 5mg/5mL, 25mg/5mL, 50mg/5mL and 100mg/5mL.

In accordance with other embodiment of the present invention, there is provided a stable suspension comprising Lamotrigine or its pharmaceutically acceptable salts, wherein the pH of suspension is in range of about 3-8. Preferably, the pH is in a range of about 4-7.

In accordance with other embodiment of the present invention, there is provided a stable suspension comprising Lamotrigine or its pharmaceutically acceptable salts, wherein the suspension is a liquid suspension packaged in a bottle.

In accordance with yet another embodiment of the present invention, there is provided a stable suspension comprising Lamotrigine or its pharmaceutically acceptable salts, wherein the suspension is a powder for suspension packaged in a bottle or sachets.

According to another embodiment of the present invention, Lamotrigine has a particle size distribution D₉₀ less than about 200 μm. Lamotrigine has a particle size distribution D₉₀ between 5 μm and 200 μm. Lamotrigine has a particle size distribution particularly D₉₀ between 5 μm and 175 μm, particularly D₉₀ between 5 μm and 150 μm, particularly D₉₀ between 5 μm and 125 μm, particularly D₉₀ between 5 μm and 100 μm and particularly D₉₀ between 5 μm and 75 μm.

In accordance with one embodiment of the present invention, there is provided a stable suspension comprising Lamotrigine or its pharmaceutically acceptable salts, wherein, the composition exhibits a comparable dissolution compared to a commercially marketed chewable dispersible tablet of Lamotrigine (Lamictal® tablet).

In accordance with one embodiment of the present invention, there is provided a process for preparation of a stable suspension comprising Lamotrigine or its pharmaceutically acceptable salts and one or more pharmaceutically acceptable excipients and/or a liquid carrier.

In accordance with one embodiment of the present invention, there is provided a process for preparation of a stable suspension comprising Lamotrigine or its pharmaceutically acceptable salts and one or more pharmaceutically acceptable excipients and/or a liquid carrier, wherein process utilized is blending, dry granulation, wet granulation, spheronization extrusion process, homogenization or the like.

In accordance with still another embodiment of the present invention, there is provided a suspension comprising Lamotrigine or its pharmaceutically acceptable esters, salts, solvates, polymorphs, enantiomers or mixtures thereof, wherein the Lamotrigine used is in amorphous form prepared by hot melt extrusion process.

In accordance with one embodiment of the present invention, there is provided a process for preparation of a ready to use suspension comprising Lamotrigine or its pharmaceutically acceptable salts and one or more pharmaceutically acceptable excipients and/or a liquid carrier, wherein process comprises the following steps:

(i) dissolving/dispersing one or more pharmaceutically acceptable excipients in a portion of water;

(ii) dispersing lamotrigine in the solution of step (i) to form a dispersion

(iii) mixing viscosity agent in another portion of water;

(iv) adding the mixture of step (iii) to the dispersion of step (ii); and

(v) optionally adding one or more pharmaceutically acceptable excipients to the dispersion of step (iv); and

(vi) optionally homogenizing the mixture of step (iv) to form a suspension.

In accordance with other embodiment of the present invention, there is provided a process for preparation of a powder for suspension comprising Lamotrigine or its pharmaceutically acceptable salts and one or more pharmaceutically acceptable excipients and/or a liquid carrier, wherein process comprises the following steps:

(i) mixing lamotrigine with one or more pharmaceutically acceptable excipients;

(ii) granulating the mixture of step (i) using a solvent;

(iii) drying the granulated mixture of step (ii);

(iv) milling the mixture of step (iii) to form granules; and

(v) mixing the granules of step (iv) optionally with one or pharmaceutically acceptable excipients to form the suspension powder for reconstitution.

In accordance with other embodiment of the present invention, there is provided a process for preparation of a powder for suspension comprising Lamotrigine or its pharmaceutically acceptable salts and one or more pharmaceutically acceptable excipients and/or a liquid carrier, wherein process comprises the following steps:

(i) mixing lamotrigine with one or more pharmaceutically acceptable excipients;

(ii) compacting the mixture of step (i) to form slugs

(iii) milling the slugs of step (ii) to form granules; and

(v) mixing the granules of step (iii) optionally with one or pharmaceutically acceptable excipients to form the suspension powder for reconstitution.

In accordance with other embodiment of the present invention, there is provided a process for preparation of a powder for suspension comprising Lamotrigine or its pharmaceutically acceptable salts and one or more pharmaceutically acceptable excipients and/or a liquid carrier, wherein process comprises the following steps:

(i) mixing lamotrigine with one or more pharmaceutically acceptable excipients; and

(ii) optionally lubricating the mixture of step (i) to form the suspension powder for reconstitution.

Powder/granules for oral suspension can be reconstituted using water or Powder/granules for oral suspension can be administered by sprinkling the powder/granules on one teaspoonful of applesauce or empty granules into a small cup or teaspoon containing one teaspoon of apple juice.

The suspension of the present invention provides advantages such as absence of lumps even after long storage when the composition is shaken as well as good pourability. The suspension of the invention has good physical stability properties such as low level of sedimentation (reduced or no caking) and easy redispersion on agitation. Moreover, it provides dose uniformity during each administration.

In accordance with another embodiment of the present invention, there is provided a ready to use liquid suspension comprising Lamotrigine or its pharmaceutically acceptable esters, salts, solvates, polymorphs, enantiomers or mixtures thereof and at least one or more pharmaceutically acceptable excipient and/or a liquid carrier comprising thickening agent/viscosity agent, antioxidant, anticaking agent, antifoaming agent, pH adjusting agent, sweetening agent, flavouring agent, solubilizer/wetting agent, buffer, and preservative wherein, the suspension is easily dispersible or re-suspendible in a pharmaceutically acceptable liquid carrier including aqueous and/or non-aqueous carrier.

In accordance with still another embodiment of the present invention, there is provided a stable suspension comprising Lamotrigine or its pharmaceutically acceptable esters, salts, solvates, polymorphs, enantiomers or mixtures thereof, wherein the composition is substantially free from other polymorphic forms.

In accordance with still another embodiment of the present invention, there is provided a suspension comprising Lamotrigine or its pharmaceutically acceptable esters, salts, solvates, polymorphs, enantiomers or mixtures thereof in an amount of about 0.01% to about 90% by weight wherein, the composition exhibits desirable technical attributes like pourability, viscosity, dissolution, stability, re-suspendability and re-dispersibility.

In another embodiment the liquid composition of the present invention includes particle size of Lamotrigine or its pharmaceutically acceptable esters, salts, solvates, polymorphs, enantiomers or mixtures thereof, having a particle size distribution such that D₉₀ is less than about 200 μm, D₅₀ is less than about 100 μm and D₁₀ is less than about 50 μm. Particularly, D₅₀ is between about 5 μm to about 100 μm. The particle size of Lamotrigine can be measured by suitable techniques such as Laser light scattering (e.g. Malvern Light Scattering), Coulter counter, microscopy, Fraunhofer diffraction and any other technique known in the art. This particle size can be obtained either by the final step during the manufacture of the Lamotrigine or by the use of conventional micronizing techniques after the crystallization procedure(s).

In another embodiment of the present invention there is provided a powder for suspension composition comprising Lamotrigine or its pharmaceutically acceptable esters, salts, solvates, polymorphs, enantiomers or mixtures thereof, present in an amount of more than 0.01% by weight based on the total weight of the composition with one or more pharmaceutically acceptable excipient and/or a liquid carrier such as thickening agent/viscosity agent, antioxidant, anticaking agent, antifoaming agent, pH adjusting agent, sweetening agent, flavouring agent, solubilizer/wetting agent, buffer, and preservative, aqueous or non-aqueous carrier and the like.

Carrier/vehicle/solvent used in the suspension of the present invention include aqueous and non-aqueous carrier but are not limited to water, alcohol, polyethylene glycol, propylene glycol or glycerin buffers, oil, or combinations thereof. Oils include peanut oil, soy bean oil, corn oil, sesame oil, cottonseed oil, acetylated glycerides, ethyl oleate, mineral oil, fatty acid esters, mono- or di- fatty acid esters of polyethylene glycols, or glyceryl mono-oleate. Particularly, the suspensions are aqueous based. By “aqueous carrier” is meant a suspension comprising water, or a combination of water and a water-miscible organic solvent or solvents. Water-miscible solvents include but are not limited to propylene glycol, polyethylene glycol and ethanol. By “non-aqueous carrier” is meant a suspension in which the carrier does not include water. The carrier can also include one more pharmaceutically acceptable excipients which can be in dissolved or dispersed form. The carrier is present in an amount from about 30 w/w % to about 95 w/w %, particularly from about 50 w/w % to about 95 w/w %.

Various useful viscosity agents/ thickening agent include, but are not limited to, gums such as xanthan gum, carrageenan gum, acacia, guar gum, locust bean gum, gum tragacanth; celluloses such as hydroxypropylcellulose (HPC), hydroxypropylmethylcellulose (HPMC), methyl cellulose, ethyl cellulose, hydroxyethyl cellulose, carboxymethylcellulose, sodium carboxymethylcellulose, mixture of microcrystalline cellulose and carboxymethylcellulose (Avicel® RC); polyvinylpyrrolidone; alginic acid; alginate; sodium alginate; bentonite; carbomers (carboxyvinyl polymers) such as those available under the trade name Carbopol®; cetostearyl alcohol; maltodextrin; polyvinyl alcohol; colloidal silicon dioxide, propylene carbonate; propylene glycol; sodium starch glycolate; starch; acrylic polymers etc. The viscosity agents are present in an amount of about 0.05% to about 20% w/w of the composition. Particularly, the viscosity agents are present in an amount of about 0.1% to about 10% w/w of the composition. More particularly, the viscosity agents are present in an amount of about 0.1% to about 5% w/w of the composition. Much more particularly, the viscosity agents are present in an amount of about 0.1% to about 3% w/w of the composition.

The suspension is easily pourable and when shaken has a viscosity in the range of 100 to 5000 cP at 25° C. Particularly, the viscosity is in the range of 100 to 2500 cP at 25° C. More particularly, the viscosity is in the range of 100 to 1500 cP at 25° C.

The term “shaken” as used herein refers to shaken prior to use, e.g. by a patient, e.g. vigorously shaken, e.g. by hand, e.g. for 5 to 40 seconds.

The viscosity can be measured by using as suitable instrument such as Brookfield viscometer, Haake VT 550 viscometer at room temperature (25° C.).

Diluents or fillers are substances which usually provide bulk to the composition. Various useful fillers or diluents include, but are not limited to sucrose, sugar alcohol, sorbitol, xylitol, erythritol, starch, pregelatinized starch, calcium carbonate, calcium phosphate, dibasic anhydrous, calcium phosphate, dibasic dihydrate, calcium phosphate tribasic, calcium sulphate, cellulose powdered, silicified microcrystalline cellulose, cellulose acetate, lactose, magnesium carbonate, magnesium oxide, maltodextrin, mannitol, microcrystalline cellulose, polydextrose, sodium alginate, sodium chloride and or mixtures thereof. Preferably diluent used is sucrose. The diluent is present in an amount of 5 to 80% of the total composition. Surfactants or surface-active agents or wetting agents or antifoaming agents improve wettability of the dosage form and/or enhance its dissolution. Also these agents help in prevention of foam formation during high shear stirring and other manufacturing process. Few active ingredients are fluffy in nature and not suspend properly. Those type of active ingredients usually float on the surface of the vehicle/ solvent used in the suspension. Surfactants or surface-active agents or wetting agents or antifoaming agent contemplated in the present invention include, but are not limited to, anionic surfactants, cationic surfactants, amphoteric surfactants, non-ionic surfactants and macromolecular surfactants. For example, polyethylene glycol stearates, poloxamer, polysorbates, sodium lauryl sulfate, dimethicone and simethicone, etc.

Various useful preservatives include, but are not limited to, parabens such as methylparaben, propylparaben, butyl paraben and their salts, sorbic acid, sodium sorbate, potassium sorbate, calcium sorbate, benzoic acid, sodium benzoate, potassium benzoate, calcium benzoate, methyl hydroxybenzoate, ethyl para-hydroxybenzoate, sodium ethyl para-hydroxybenzoate, sodium metabisulphite, chlorhexidine, diazolidinyl urea, sodium citrate, butylated hydroxyl toluene (BHT), butylated hydroxyl anisole (BHA), tocopherol, ethylenediamine tetraacetic acid, propyl gallate, quaternary compounds, e.g. benzalkonium chloride and cetylpyridinium chloride, phenyl ethyl alcohol and the like. In particular, the preservative is selected from benzoic acid and its salts and parabens. The preservative is present in an amount of about 0.001% w/w to about 3% w/w.

Anticaking agent helps to re-suspend the ingredients suspended in the formulation. Generally, suspension formulations contain micronized particles of active ingredients and inactive ingredients, which settle at the bottom of the container and form a thin hard cake, which is not easily re-suspendable after shaking. Anticaking agent helps to improve the re-suspendability of the formulation. Various useful Anticaking agents include, but are not limited to, colloidal silica and/or colloidal silicon dioxide, calcium phosphate tribasic, magnesium oxide, magnesium silicate, calcium silicate or the like.

Various useful antioxidants include, but are not limited to, ascorbic acid, tert-butylhydroquinone, sodium pyrosulfite, glutathione, sodium bisulfite, sodium sulfite, a-tocopherol, a-tocopherol acetate, monothioglycerol, cysteine, ascorbyl palmitate, acetylcysteine, dithiothreitol, sodium metabisulfite, thiourea, sodium thiosulfate, butylated hydroxy anisole (BHA), butylated hydroxytoluene (BHT) and propyl gallate.

Various useful sweetening agents include, but are not limited to, sugar or a sugar alcohol such as sucrose, dextrose, sucralose, sorbitol, fructose, mannitol and invert sugar and sugar substitutes such as saccharin sodium, aspartame. Sugar or a sugar alcohol can also act as filler. Preferably sweetening agent used is sodium saccharin. Various useful flavoring agents, include, but are not limited to, flavors such as banana, lemon, orange, grape, lime and grapefruit, vanilla, and fruit essence, including apple, banana, pear, peach, strawberry, raspberry, cherry, plum, pineapple, apricot; synthetic flavor oils and flavoring aromatics and/or natural oils, extracts from plant leaves, flowers, fruits such as cinnamon oil, oil of wintergreen, peppermint oils, clove oil, citrus oil, bay oil, anise oil, eucalyptus, thyme oil, cedar leaf oil, oil of nutmeg, oil of sage, oil of bitter almonds, and cassia oil; maltol, ethyl vanillin, menthol, citric acid, fumaric acid, ethyl maltol, and tartaric acid and combinations thereof.

Various useful isotonizing agent include, but are not limited to, sodium chloride, mannitol, D-sorbitol, glucose, glycerin or the like.

Various useful pH adjusting agent or buffering agents include, but are not limited to, citrate buffers, phosphate buffers, or any other suitable buffer known in the art including monosodium dibasic phosphate, gluconic acid, lactic acid, citric acid, acetic acid, sodium gluconate, sodium lactate, sodium citrate, sodium acetate potassium citrate, sodium bicarbonate, potassium bicarbonate, sodium dihydrogen phosphate and potassium dihydrogen phosphate.

Various useful taste masking agents include, but are not limited to, water soluble and/or insoluble polymeric excipient, water insoluble non-polymeric excipient, adsorbent, ion exchange resin, carbomer, alkali metal chlorides or an alkaline earth metal chlorides or a derivative thereof.

The pharmaceutical composition of the present invention can be packaged in a suitable pack/container such as amber colored polyethylene terephthalate (PET) bottle, glass bottle, high density polyethylene (HDPE) bottle, low density polyethylene (LDPE) bottle, polypropylene (PP) bottle, packets, pouches, sachets and the like. The glass or plastic bottle is provided with a child proof closure. The package can include a syringe (marked in mL) for ease of dosing.

The container such as bottle has a fill volume of, e.g., from about 50 mL to about 500 mL comprising lamotrigine suspension. Pack chosen are made of material which is non-reactive with the suspension and suspension powder for reconstitution. Containers for use in the storage of the oral suspensions may be used to administer a multiple dose of lamotrigine.

The liquid pharmaceutical composition of the present invention can be used for treatment of epilepsy, bipolar disorder, partial seizures, primary and secondary tonic-clonic seizures, and seizures associated with Lennox-Gastaut syndrome.

Having described the invention with reference to certain preferred embodiments, other embodiments will become apparent to one skilled in the art from consideration of the specification. The invention is further defined by reference to the following examples describing in detail method for the preparation and testing of Lamotrigine pharmaceutical composition. It will be apparent to those skilled in the art that many modifications, both to materials and methods, may be practiced without departing from the scope of the invention. Following examples are set out to illustrate the invention and do not limit the scope of the present invention.

### EXAMPLES

TABLE 1
Ready to use suspension

% (w/v)

| Ingredients | Example 1 | Example 2 |
|---|---|---|
| Lamotrigine | 0.1-4 | 1.2 |
| Sucrose | 5-60 | 13 |
| Monosodium dibasic | 0.02-0.05 | 0.03 |
| phosphate | | |
| Sorbitol | 10-40 | 40 |
| Sodium saccharin | 0.01-0.05 | 0.020 |
| Avicel RC 591 | — | 0.5 |
| (Microcrystalline cellulose | | |
| and carboxy methyl | | |
| cellulose sodium) | | |
| Xanthan gum | 0.1-0.8 | 0.3 |
| Banana Flavor | 0.01-0.9 | — |
| Pineapple Flavor | — | 0.15 |
| Sodium Benzoate | 0.08-0.4 | — |
| Potassium sorbate | — | 0.10 |
| Methyl paraben | 0.1-1.0 | 0.25 |
| Propyl paraben | 0.05-1.0 | 0.05 |
| Water | Up to 100 | Up to 100 |

Procedure:

1. Sucrose and sorbitol were added to the hot water to form a solution.

2. Lamotrigine was dispersed in the solution of step 1.

3. Xanthan gum and Avicel RC 591 were mixed in another portion of water and added to the dispersion of step 2.

4. Sodium saccharin, mono sodium dibasic phosphate, potassium sorbate and/or sodium benzoate, methyl paraben, propyl paraben and desired flavor were added under stirring to the dispersion of step 3.

5. The dispersion of step 4 was homogenized and the volume was made up with the remaining quantity of water to form the suspension.

TABLE 2
Powder for suspension

| | % (w/w) | | |
|---|---|---|---|
| Ingredients | Example 3 | Example 4 | Example 5 |
| Lamotrigine | 0.1-8 | 2 | 4.00 |
| Sucrose | 40-80 | 66 | 65 |
| Monosodium dibasic | 0.03-0.07 | 0.1 | 0.05 |
| phosphate | | | |
| Sorbitol | 10-40 | 30 | 30.00 |
| Sodium saccharin | 0.03-0.20 | 0.1 | 0.04 |
| Avicel RC 591 | 0.0-1.2 | 0.7 | 0.70 |
| (Microcrystalline cellulose | | | |
| and carboxy methyl | | | |
| cellulose sodium) | | | |
| Xanthan gum | 0.10-0.90 | 0.75 | 0.45 |
| Banana flavor and/or | 0.05-0.3 | — | 0.2 |
| Cherry Flavour | | | |
| (optional) | | | |
| Potassium sorbate | 0.05-0.5 | 0.25 | 0.15 |

Procedure:

1. Lamotrigine, sucrose, monosodium dibasic phosphate anhydrous, sorbitol powder were mixed.

2. Sodium saccharin was dissolved in water.

3. The mix of step 1 was granulated with solution of step 2.

4. The wet mass of step 3 was dried and milled to form granules.

5. Avicel RC 591, xanthan gum, desired flavors were mixed and blended with the granules of step 4 to form the suspension powder for reconstitution.

The suspension powder for reconstitution was reconstituted with water to provide a strength of 10 mg/mL.

As is generally known by those skilled in the art, following tests were performed in reference with USP, Remington: The Science and Practice of Pharmacy 20th Edition and L. Lachman, H. A. Lieberman, J. L. Kanig (1986).

Assay for Lamotrigine

The ready to use suspension of Example 2 and powder for suspension of Example 4 after reconstitution were analyzed for drug content by HPLC as per method disclosed in USP <621>and the results are provided in Table 3.

TABLE 3
Assay for Lamotrigine

| Composition | % Assay |
|---|---|
| Example 2 | 100.1 |
| Example 4 | 106.0 |

pH data: pH values were determined using potentiometry using USP <791>The pH of the reconstituted suspension of Example 4 was determined to be 5.6. The pH of the ready to use suspension of Example 2 was determine to be 5.56

Viscosity data: The viscosity of the reconstituted suspension of Example 4 was determined to be 1029 cps using Brookfield viscometer with spindle Sc4-18 and rpm 2 at 25° C. The viscosity of ready to use suspension of Example 2 with the same method was found to be 800cps.

Microbiological Studies: Microbial examination of non-sterile product was performed according to the methods given in the text on microbiological examination of non-sterile products: Microbial Enumeration Test <61>and Test for Specified Microorganism <62>. Limit content of microorganisms was performed according to the criteria given in the texts: <1111>USP-30 NF-25.

The powder for oral suspension of Example 5 shows no microbial growth for at least 20 days when examined as per the above mentioned method.

TABLE 4
Microbiological studies

| | | | | | Microbial |
|---|---|---|---|---|---|
| Condition | Period | Pack | TAMC* | TYMC^{#} | Growth |
| 25° C./60% RH | 20 Days | Glass Bottle | <100 cfu/mL | 10 cfu/mL | Absent |
| 2-8° C. | 20 Days | Glass Bottle | <100 cfu/mL | <10 cfu/mL | Absent |

*TAMC: Total aerobic microbial count
^{#}TYMC: Total yeast and mould count
Cfu: Colony Forming Units

Dissolution Studies

The ready to use suspension of Example 2 and powder for suspension of Example 4 were evaluated for in-vitro lamotrigine release. The in-vitro dissolution was determined using a USP type II apparatus at 50 rpm in 900 mL of 0.1N HCL at 37±0.5° C. The results are represented in Table 5.

TABLE 5
Percentage (%) of the In-Vitro Lamotrigine Release in USP type II
apparatus at 50 rpm in 900 mL of 0.1N HCL

| | Percentage of Lamotrigine Release | | |
|---|---|---|---|
| | | | Lamictal Chewable |
| Time (minutes) | Example 2 | Example 4 | Dispersible Tablet 25 mg |
| 5 | 55 | 56 | — |
| 10 | 69 | 76 | 102 |
| 15 | 79 | 89 | 101 |
| 20 | 87 | 92 | 101 |
| 30 | 98 | 93 | 101 |
| 45 | 102 | 94 | 101 |

1. An immediate release oral pharmaceutical powder for suspension of lamotrigine or its pharmaceutically acceptable salt, hydrate or polymorph thereof with one or more pharmaceutically acceptable excipients comprising lamotrigine present at from about 0.1% w/w to 40% w/w of the power for suspension, wherein the pH of the reconstituted composition is from 4-8 when reconstituted with water.

2. The immediate release oral pharmaceutical powder for suspension of claim 1, wherein the pH of the reconstituted composition is from 4-8 and the viscosity is from 700-1200 cps.

3. The immediate release oral pharmaceutical powder for suspension of claim 1, wherein the composition is free from microbial contamination for at least 20 days when tested according to <1111>USP-30 NF-25.

4. An immediate release oral pharmaceutical powder for suspension of lamotrigine or its pharmaceutically acceptable salt, hydrate or polymorph thereof with one or more pharmaceutically acceptable excipients comprising lamotrigine from about 0.01% w/w to 10% w/w by weight on the basis of the total weight of the composition wherein the composition exhibits in-vitro dissolution rate of more than 85% of drug release within 20 minutes when placed in a dissolution vessel filled with 900 ml of 0.1N HCL, pH 1.2 maintained at 37±0.5° C. and stirred at a paddle speed of 50 rpm using a USP Type II (paddle) apparatus.)

5. The immediate release oral pharmaceutical powder for suspension of claim 1, wherein the one or more pharmaceutically acceptable excipients comprise:
a) one or more thickening/viscosity agents selected from gums and/or celluloses;
b) one or more diluents and/or sweetening agents selected from sugars and sugar alcohols;
c) one or more preservatives;
d) one or more antioxidants;
e) one or more pH adjusting agents to maintain the pH of the reconstituted composition in the range of about 4.0 to about 8.0; and/or a pharmaceutically acceptable liquid carrier for reconstitution;
wherein the composition is free of a glidant.

6. The immediate release oral pharmaceutical powder for suspension of claims 5, wherein the thickening/viscosity agent comprise xanthan gum from about 0.13 to 1.0% w/w and the diluent comprises sucrose from about 40 to 80% w/w.

7. (canceled)

8. The immediate release oral pharmaceutical powder for suspension of claim 1 prepared by a process comprising the following steps:
i. mixing lamotrigine with one or more pharmaceutically acceptable excipients;
ii. granulating the mixture of step (i) using a solvent;
iii. drying the granulated mixture of step (ii);
iv. milling the mixture of step (iii) to form granules; and
v. mixing the granules of step (iv) optionally with one or pharmaceutically acceptable excipients to form the suspension powder for reconstitution.

9. An immediate release oral pharmaceutical suspension of lamotrigine or its pharmaceutically acceptable salt, hydrate or polymorph thereof with one or more pharmaceutically acceptable excipients prepared by a process comprising the following steps:
(i) dissolving/dispersing one or more pharmaceutically acceptable excipients in a portion of water;
(ii) dispersing lamotrigine in the solution of step (i) to form a dispersion
(iii) mixing viscosity agent in another portion of water;
(iv) adding the mixture of step (iii) to the dispersion of step (ii);
(v) optionally adding one or more pharmaceutically acceptable excipients to the dispersion of step (iv); and
(vi) optionally homogenizing the mixture of step (iv) to form a suspension.

10. The immediate release oral pharmaceutical composition of claim 1 comprising lamotrigine having a particle size distribution D₉₀ less than about 20 m, and wherein the composition is prepared using dry granulation, wet granulation, blending, spheronization extrusion process, homogenization and/or holt melt extrusion processes.

11. The oral pharmaceutical powder for suspension of lamotrigine or its pharmaceutically acceptable salt, hydrate or polymorph thereof of claim 1, wherein the powder for suspension comprises:
a) lamotrigine from about 0.1% w/w to 40% w/w,
b) one or more thickening/viscosity agents selected from gums and/or celluloses from about 0.1% w/w to 10% w/w,
c) one or more diluents and/or sweetening agents selected from sugars and sugar alcohols from about 5% w/w to 80% w/w,
d) one or more preservatives from about 0.001% w/w to 3% w/w,
e) one or more antioxidants, and
f) one or more pH adjusting agents.

12. The oral pharmaceutical powder for suspension of lamotrigine or its pharmaceutically acceptable salt, hydrate or polymorph thereof of claim 4, wherein the powder for suspension comprises:
a) lamotrigine from about 0.1% w/w to 40% w/w,
b) one or more thickening/viscosity agents selected from gums and/or celluloses from about 0.1% w/w to 10% w/w,
c) one or more diluents and/or sweetening agents selected from sugars and sugar alcohols from about 5% w/w to 80% w/w,
d) one or more preservatives from about 0.001% w/w to 3% w/w,
e) one or more antioxidants, and
f) one or more pH adjusting agents.
